(19) United States
(12) Patent Application Publication
(10) Pub. No.: US 2014/0374623 A1
(43) Pub. Date: Dec. 25, 2014

(54) METHOD AND APPARATUS FOR MEASURING OPTICAL PROPERTIES OF PARTICLES OF A DISPERSION

(71) Applicant: Partec GmbH, Goerlitz (DE)

(72) Inventor: Wolfgang Goehde, Nottuln (DE)

(21) Appl. No.: 14/481,560

(22) Filed: Sep. 9, 2014

Related U.S. Application Data
(63) Continuation of application No. 13/673,207, filed on Nov. 9, 2012, now abandoned.
Application No. 13/673,207 is a continuation of application No. PCT/DE2011/075110, filed on May 13, 2011.

(30) Foreign Application Priority Data
May 18, 2010 (DE) 10 2010 017015.1

(51) Int. Cl. G01N 21/64; G01N 33/487; G01N 15/14

(52) U.S. Cl. CPC G01N 21/6486; G01N 15/1434; G01N 33/487; G01N 2201/06113
USPC 250/459.1

(57) ABSTRACT

A method for measuring optical properties of particles of a flowable dispersion using a measuring cuvette. As the dispersion flows through the flow chamber of the cuvette, two laser light beams, offset 90 degrees to one another, illuminate the inner chamber of the cuvette and excite fluorescence in a particle. Regardless of the orientation of the particle, the total fluorescing light of the particle provides an accurate measurement of the contents of the cell and balances out form factor errors.

### BACKGROUND INFORMATION

1. Field of the Invention

The invention relates to the field of cytology. More particularly, the invention relates to improved cytometric analysis apparatus.

2. Discussion of the Prior Art

Precise measurement of optical properties of a large number of particles of a flowable dispersion, which can be gaseous or liquid, is very important in cytology and when considering technical problems. Currently, flow cytometers allow photometric and fluorescent-optical analysis of several thousand particles per second. Furthermore, devices have been introduced that make it possible to sort the desired particles or cells online based on the previous measurements. Such an arrangement of flow cytometers with downstream cell sorting serves, for example, to separate sperm containing X and Y chromosomes for further use in animal breeding.

For cell sorting, see, e.g., the publication by Bessette, P. H. and Daugherty, P.S. (2004), Flow Cytometric Screening of cDNA Expression Libraries for Fluorescent Proteins. Biotechnology Progress, 20:963967. doi:10.1021/bp034308g.

A device used by the applicant for cell sorting is known in the field under the name “Particle and Cell Sorter PPCS”, wherein the operating method of this device is described in the associated manual.

Precise measurement of optical properties of particles is made more difficult, when the particles have very different shapes and sizes.

Numerous important application areas for flow cytometers are found in biotechnology, production process monitoring, cell analysis, cytopathology, and immunology.

The goal of cytometric analysis is to quickly detect individual particles and to obtain the most precise possible optical measurement of the particles. Generally, lasers are used as light sources for forward and sidewise diffused light measurements, as well as for fluorescence excitation. The laser light impinges on the particles at a very small aperture angle (low divergence) or as a parallel light beam. The technical measurement problem herein lies in the fact that the laser beam hits the morphologically complex particles, which are often structured as very flat, two-dimensional objects, at a right angle to the particle surface, i.e., hits the flat surface, or parallel to the particle surface, i.e., hits the edge. This results in a “correct” measurement of the particle fluorescence when the laser beam illuminates the surface and an “incorrect” measurement when the laser beam hits the edge of the particle.

The flow cytometrical analysis of microscopically small particles has significant economic meaning, if highly precise measurements are obtained. One application, for example, is measuring the DNA content of sperm. If this measurement is precise enough, the two types of sperm containing the X and Y chromosomes can be sorted with a device downstream from the flow cytometer, something that is very useful in animal breeding.

The difference in the DNA content of these two types of sperm is very small. In cattle, for example, the difference amounts to only 1.6% (total amount of DNA per cell: 3.3 pg), and very precise measurement of the cells is required in order to distinguish them reliably.

One suggestion has been to orient the cells, before the passing of the laser beam exciting the fluorescence, such that the cells are preferably hit by the laser beam on their surface. This orientation is achieved more or less only by a flat or elliptical outlet opening in the tube that guides the cell suspension to the measuring area.

Another way of avoiding the “orientation error” was achieved by illuminating the cells for fluorescence excitation with an extremely large numerical aperture (large solid angle). This has previously been accomplished only by using conventional light sources, e.g., discharge lamps. With this method, every cell is evenly illuminated on all sides, which largely eliminates the form factor.

This manner of illumination with a large numerical aperture certainly reduces the form-factor influence, but it does not permit the detection of other, often more important, parameters of the cells, such as, forward diffused light (particle size) or sidewise diffused light (homogeneity factor).

### BRIEF SUMMARY OF THE INVENTION

The object of the invention is to reduce or completely exclude the erroneous influence of form factors on the measurement precision in conventional laser-based flow cytometers. Reducing the form-factor error results in a significantly greater precision of the measurement. It is also a goal of the invention to combine the greater measurement precision with the two scatter light parameters, i.e., particle size and homogeneity, as these cannot be measured with conventional light sources. The device for laser photoexcitation according to the invention avoids the influence of form factors. Compared to conventional light sources with the advantage of excitation with high numerical aperture, the laser excitation permits a much higher light energy density, which results in more precise measurements, because of a better signal-to-noise ratio.

### BRIEF DESCRIPTION OF THE DRAWINGS

The present invention is described in greater detail with reference to the purely schematic drawings.

FIG. 1 is a schematic cross-sectional view of a flow cytometer;

FIG. 2 is a top view of a first embodiment of a flow cytometer;

FIG. 3 illustrates views from two different directions of two different cell types that are to be measured;

FIG. 4 is a perspective view of a second embodiment;

FIG. 5 is a top view of a third embodiment of a flow cytometer; and

FIG. 6 is a top view of a fourth embodiment of a flow cytometer.

### DETAILED DESCRIPTION OF THE INVENTION

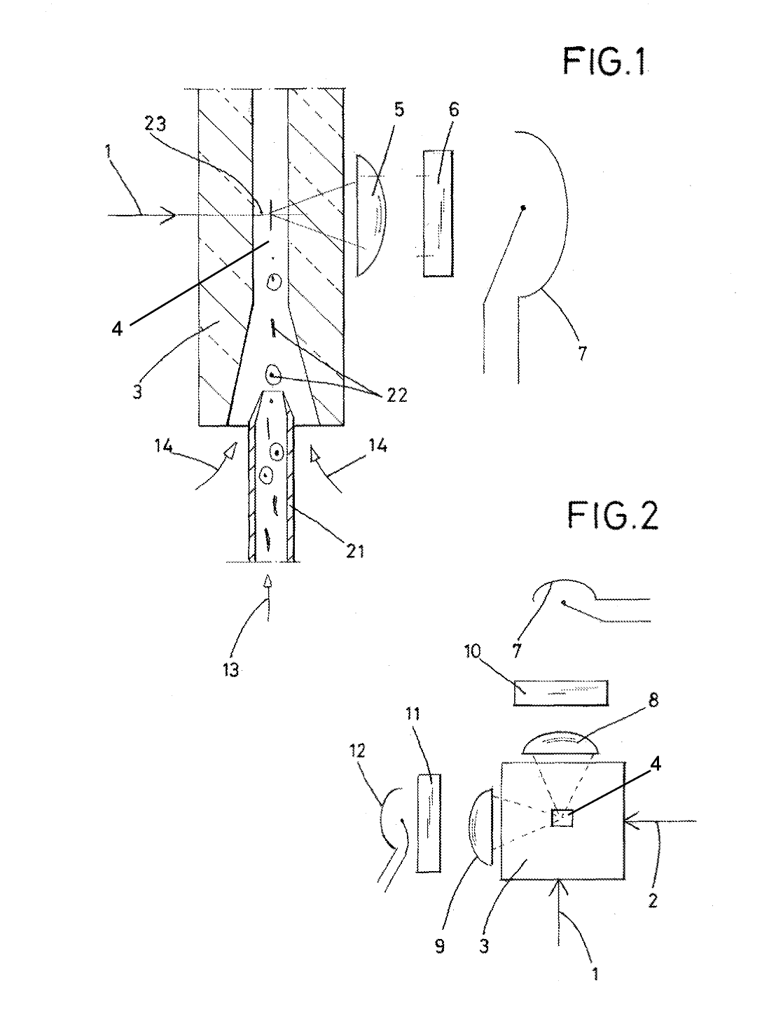

FIG. 1 is a schematic cross-sectional vertical cut through the cuvette portion of a flow cytometer, showing a flow channel 4, the excitation beam path, using a laser unit that emits a laser beam 1, and the measurement beam path with light-collecting optics in the form of a collecting lens 5, a laser-light blocking filter 6, and a light-sensitive sensor 7, also referred to as a photodetector. The particle dispersion 13 is fed into the flow channel 4 in the measuring cuvette 3 through a narrow tube 21. A plurality of particles P are shown in the flow channel 4.

A particle-free medium 14 is fed around the particle stream 13, resulting in a centering of the particles when passing the measuring area at 23. The morphologically different particles have no preferred orientation.

FIG. 2 and FIGS. 4-6, are schematic illustrations that show a top view or a perspective view of the measuring cuvette 3. These illustrations make clear that, according to the invention, laser photoexcitation occurs from two directions, whereby the beaming direction of laser light beam 1 is offset by 90° to that of laser light beam 2. Although not shown, it is understood that the light beam emitted from the laser unit may be split into a first laser light beam 1 and a second laser light beam 2. The use of the two offset light beams 1 and 2 results in “flat” particles being illuminated edgewise by laser 1 and on the wide flat area by laser 2, or vice versa, depending upon the orientation of the two-dimensional particles in the flow channel 4. The particles can have any orientation, but the total amount of light from the two laser light beams 1 and 2 that reaches the fluorescent material is constantly the same.

In the embodiment in FIG. 2, the laser light, in the form of the two laser light beams 1 and 2, excites fluorescence from two sides of the cuvette 3. In addition to the light-collecting lens, here identified as a first collecting lens 8, a second light-collecting optic or collecting lens 9 is mounted so as to be offset by 90 degrees to the first collecting lens 8. Capturing the light emitted from every particle in this way reduces even more the influence of form factors. Both beam paths for capturing light are equipped with optics having high numerical apertures, in the form of collecting lenses 8 and 9, as well as with blocking filters 10 and 11 for the exciting laser light, and with photodetectors 7 and 12.

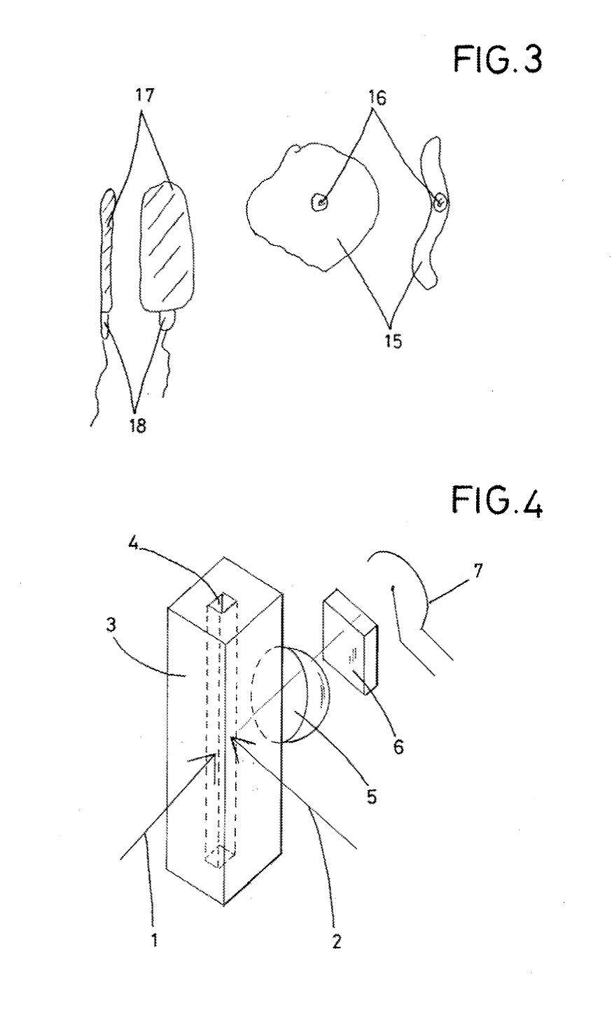

FIG. 3 shows an epithelial cell, which can have a diameter of up to 60 μm in humans. If the cell is illuminated on edge, only weak laser light reaches the nucleus 16. But, if the cell is beamed two-dimensionally, i.e., on its flat surface, and edgewise, the beaming errors are cancelled out.

This problem becomes even more apparent when measuring sperm. The substance to be measured, the DNA, is found in sperm head 17, and RNA is preferably found in the middle portion 18. If such an object is beamed edgewise, the laser light does not reach all parts of the DNA evenly; a portion of the excitation light is “scattered off”, and light absorption occurs inside of sperm head 17, which means that not all parts of the DNA are equally fluorescence-excited.

FIG. 4 shows the arrangement with laser double-excitation in a perspective drawing. The drawing shows schematically, by way of example, just the measurement optic, which includes the collecting lens 5, the laser light blocking filter 6, and the photodetector 7. The collecting lens 5 has a high numerical aperture, so that the influence of form factors is avoided on the measuring side. Alternative to the depicted embodiment, each of the laser light beams 1 and 2 may be provided with its own measurement optic, as shown in the embodiment in FIG. 2.

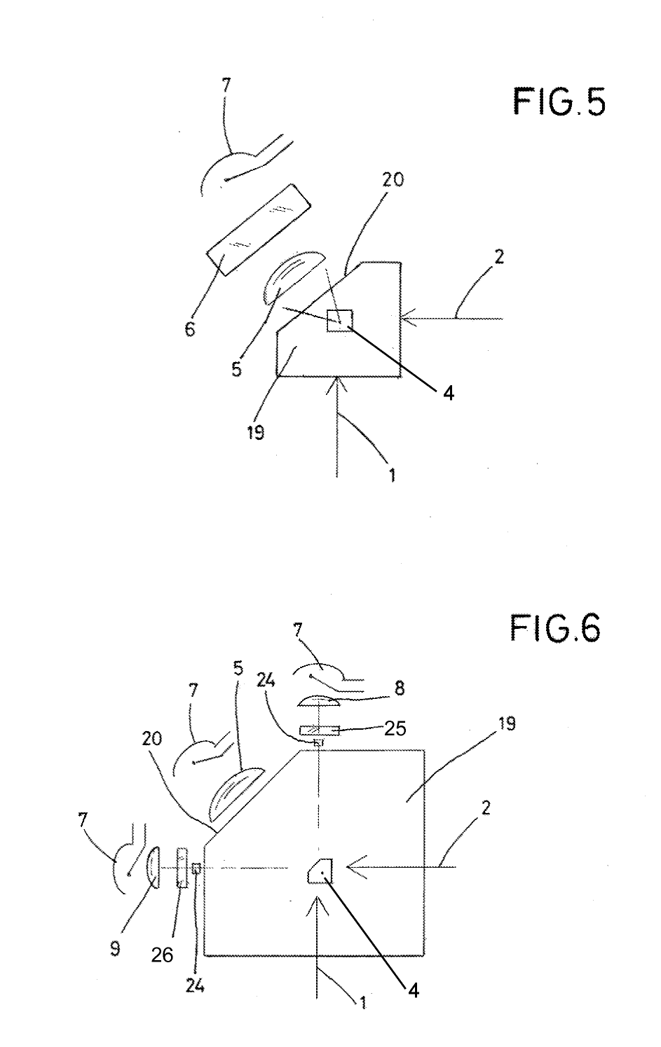

FIG. 5 shows another embodiment of the measurement apparatus according to the invention: Because the available laser light blocking filters 6, 10, and 11 do not completely block out the exciting laser light at some wavelengths, an arrangement is shown in FIG. 5 that prevents the respective laser light beams 1 and 2 from reaching the measuring optic in a direct path.

The optical axis of the connecting lens 5 is also arranged vis-à-vis the beam directions of the two excitation laser light beams 1 and 2 in a way that halves their angles, that is, at an angle of either 45° and 135°, respectively. A special construction of the measuring cuvette 19, with a fifth surface drawn as a diagonal surface 20 in the cross-section, is provided for this. This fifth surface allows the arrangement of a measuring optic with high numerical aperture, without laser excitation light getting into the measuring beam path. The parallel orientation of the front surface of the collecting lens 5 to the diagonal surface 20 guarantees the fluorescent light radiates from the measuring cuvette 19 into the collecting lens 5 with low loss.

FIG. 6 shows a measuring cuvette that is essentially equipped like the one in FIG. 5 and therefore also identified with 19. Due to its two surfaces, which are arranged at a 90° angle to each other and border the diagonal surface 20, in addition to the collecting lens 5, which is allocated to the diagonal surface 20, two collecting lenses 8 and 9 arranged at an angle of 90° may also be provided, similar to the depiction shown in FIG. 2. Especially precise measuring results may be obtained with this arrangement.

The photodetectors 7, which are behind the collecting lenses 8 and 9, do not evaluate the fluorescent light, but rather the forward diffused light or the negative absorption light, which results after the excitation light, that is, the laser light beams 1 and 2, hits the respective cells. Blocking filters 25 and 26 are provided between the measuring cuvette 19 and the collecting lenses 8 and 9. They block undesirable light components and are permeable, as much as possible, only for the diffused light. Since only diffused light components are to be captured, that is, incident light components at an angle of more than 0 degrees, the direct-incidence laser light is blocked with so-called laser stops 24.

The orientation of the cells inside the measuring cuvette 19 can be calculated from the two forward diffused light signals, so that, for example, the measurements of cells that are unfavorably oriented are not taken into account for further analysis, or so that certain correction factors can be allocated to the measurements of the fluorescent light, depending upon the orientation of the measured cell.

In the measuring arrangement according to FIG. 6, only the fluorescent light emitted by the examined cells is captured by the collecting lens 5, because the laser light beams 1 and 2 are not aimed at the collecting lens 5. This arrangement allows a particularly clear measurement signal to be received, one that is not contaminated by laser light components. For this reason, shown purely as an example, no laser-light blocking filter is provided between the diagonal surface 20 and the photodetector 7 that is allocated to that surface. A laser light blocking filter 6, as in FIG. 5, may also be provided to shut out undesirable diffused light components.

In an alternative embodiment, provision may be made to guide the light from the measuring cuvette in the direction of the photodetector through an optical element that is constructed as a cylinder having a cylindrical reflection surface. In this case, the cylinder may be constructed as a hollow cylinder whose inner surface forms the cylindrical reflection surface or as a solid, light-permeable cylinder, the outer surface of which forms the cylindrical reflection surface.

It is understood that the embodiments described herein are merely illustrative of the present invention. Variations in the apparatus for the optical measurement of particles in a flowable dispersion may be contemplated by one skilled in the art without limiting the intended scope of the invention herein disclosed and as defined by the following claims.

What is claimed is:

1. A method of reducing an influence of form factors in a measurement of cell contents of particles in a particle dispersion, the method comprising:
a) providing a flow cytometer having a flow chamber, an excitation beam path, and a measurement beam path;
b) feeding a flow of the particle dispersion through the flow chamber;
c) centering the particle dispersion along the measurement beam path;
d) exciting fluorescence from particles in the particle dispersion flowing through the measurement beam path, each of the particles being simultaneously illuminated by photo-excitation apparatus from two excitation directions, each excitation direction offset by 90 degrees from the other excitation direction; and
e) obtaining an accurate measurement of the cell contents based on a calculation of the light emitted from the fluorescing particles.

2. The method of claim 1, wherein the step of centering the particle dispersion includes feeding a particle-free medium through the flow chamber, such that the particle-free medium surrounds the particle dispersion.

3. The method of claim 1, wherein the excitation beam path includes a first laser beam and a second laser beam, each beam offset from the other by 90 degrees, and wherein each beam hits a particle in the flow chamber simultaneously.
